(19) United States
(12) Patent Application Publication
(10) Pub. No.: US 2014/0248229 A1
(43) Pub. Date: Sep. 4, 2014

(54) NOURISHING HAIR TREATMENT COMPOSITION

(71) Applicant: Henkel AG & Co. KGaA, Duesseldorf (DE)

(72) Inventor: Marcus Krueger, Ellerhoop (DE)

(21) Appl. No.: 14/273,708

(22) Filed: May 9, 2014

Related U.S. Application Data
(63) Continuation of application No. PCT/EP2012/068728, filed on Sep. 24, 2012.

(30) Foreign Application Priority Data
Nov. 11, 2011 (DE) 10 2011 086 218.8

(51) Int. Cl. A61K 8/84; A61Q 5/02; A61Q 5/00

(52) U.S. Cl. CPC A61K 8/84; A61Q 5/00; A61Q 5/02
USPC 424/70.17

(57) ABSTRACT

Hair treatment compositions, including at least one quaternary imidazoline derivative having at least two long fat residues of the formula (I) set forth herein, in which the R¹ and R² residues are each independently a saturated or unsaturated, linear or branched hydrocarbyl residue having a chain length of 18 to 30 carbon atoms and X— is an anion, at least one amine and/or a cationized amine and at least one esterquat, are enhanced when they additionally include poly(2-methacryloyloxyethyltrimethylammonium chloride) (INCI name: Polyquaternium-37) to improve combability, shine and elasticity.

### FIELD OF THE INVENTION

The present invention generally relates to cosmetic hair treatment agents based on a special active-agent combination, to a method using the agents, and to use of the agents in order to care for and improve the combability of keratinic fibers.

### BACKGROUND OF THE INVENTION

The significance of care-providing products having the longest-lasting effect possible is increasing, not least due to severe stress on hair as a result, for example, of coloring or permanent waving, and also as a result of cleaning the hair with shampoos and because of environmental stresses.

The known active agents cannot, however, meet all needs to a sufficient extent. A demand therefore continues to exist for active agents or active-agent combinations for cosmetic agents having good care-providing properties and good biodegradability. In dye- and/or electrolyte-including formulations in particular, a demand exists for additional care-providing active agents that can be incorporated without difficulty into known formulations.

Hair treatment agents that include at least one quaternary imidazoline derivative having at least two long fat residues, at least one amine and/or cationized amine, and at least one esterquat are known from WO 2010/000569 A1.

It is therefore desirable to refine this active-agent combination and improve it in particular in terms of the combability and softness (feel) of keratinic fibers.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair treatment agent including at least one quaternary imidazoline derivative having at least two long fat residues in accordance with formula I

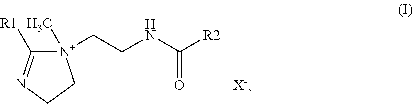

in which the residues R¹ and R² mutually independently each denote a saturated or unsaturated, linear or branched hydrocarbon residue having a chain length from 18 to 30 carbon atoms, and X⁻ denotes an anion; at least one amine and/or cationized amine; at least one esterquat, characterized in that it additionally includes poly(2-methacryloxyethyltrimethylammonium chloride) (INCI name: Polyquaternium-37).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that the addition of specific polymers to the aforementioned combination of three ingredients improves combability values, shine, and elasticity even further. In addition, it was also possible to improve the washing fastness of colored hair. Surprisingly, the softness of the keratinic fibers in particular was extraordinarily positively influenced.

The subject of the present invention is, in a first embodiment, hair treatment agents including
a) at least one quaternary imidazoline derivative having at least two long fat residues in accordance with formula I

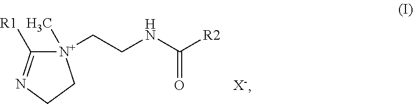

in which the residues R¹ and R² mutually independently each denote a saturated or unsaturated, linear or branched hydrocarbon residue having a chain length from 18 to 30 carbon atoms, and X⁻ denotes an anion,
b) at least one amine and/or cationized amine,
c) at least one esterquat,
   which additionally include
d) poly(2-methacryloxyethyltrimethylammonium chloride) (INCI name: Polyquaternium-37).

“Hair treatment agents” for purposes of the present invention are, for example, hair coloring agents, hair-bleaching agents, hair shampoos, hair conditioners, conditioning shampoos, hair sprays, hair rinses, hair therapies, hair packs, hair tonics, permanent-wave setting solutions, hair coloring shampoos, hair coloring agents, hair setting agents, hair setting compositions, hair styling preparations, blow-dry lotions, foam setting agents, hair gels, hair waxes, or combinations thereof. Preferred agents according to the present invention are shampoos, conditioning agents, or hair tonics.

The agents according to the present invention include as ingredient a) at least one quaternary imidazoline compound of formula I, i.e. a compound that comprises a positively charged imidazoline ring.

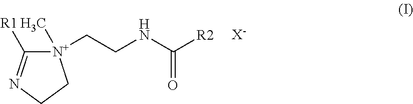

Residues R¹ and R² mutually independently each denote a saturated or unsaturated, linear or branched hydrocarbon residue having a chain length from 18 to 30 carbon atoms.

The agents according to the present invention preferably include compounds of formula (I) in which R¹═R². The chain length of the residues R is at least 18 carbon atoms. Compounds having a chain length of at least 19 carbon atoms are preferred, and those having 20 carbon atoms are particularly preferred. A very particularly preferred compound of formula I has a chain length of 21 carbon atoms. A commercial product of this chain length is known, for example, by the name Quaternium-91.

The counter ion X— in formula (I) is preferably methosulfate, although the halides such as chloride, fluoride, bromide, or also phosphates, are also suitable as counter ions.

The imidazolines of formula (I) are included in the compositions according to the present invention preferably in quantities from 0.01 to 20 wt %, preferably in quantities from 0.01 to 10 wt %, and very particularly preferably in quantities from 0.1 to 7.5 wt %. The best results of all are obtained with quantities from 0.1 to 5 wt %, based in each case on the total composition of the respective agent.

Very particularly preferred hair treatment agents according to the present invention are characterized in that they include, based on their weight, 0.1 to 15 wt %, preferably 0.2 to 10 wt %, more preferably 0.3 to 7.5 wt %, even more preferably 0.4 to 5 wt %, and in particular 0.5 to 2.5 wt % of at least one quaternary imidazoline derivative of formula I in which R¹ and R² each denote —(CH₂)₂₀—CH₃ and X⁻ denotes CH₃—OSO₃⁻.

The agents according to the present invention include as ingredient b) at least one amine and/or cationized amine. Preferred compounds of this substance class are amidoamines and/or cationized amidoamines, compounds of the following formulas (II) and/or (III) being preferred:

R¹—NH—(CH₂)ₙ—NR²R³ (II) and/or

R¹—NH—(CH₂)ₙ—NR²R³R⁴ (III),

in which R¹ signifies an acyl or alkyl residue having 6 to 30 carbon atoms which can be branched or unbranched, saturated or unsaturated, and wherein the acyl residue and/or the alkyl residue can include at least one OH group, and
R², R³, and R⁴ mutually independently in each case can be hydrogen or an alkyl residue having 1 to 4 carbon atoms, which can be identical or different, saturated or unsaturated, and
X⁻ signifies an anion, and
n signifies an integer between 1 and 10.

The anion is selected from the physiologically acceptable anions, Examples thereof that may be recited are the halide ions fluoride, chloride, bromide, sulfates of the general formula RSO₃⁻ in which R has the meaning of a saturated or unsaturated alkyl residue having 1 to 4 carbon atoms, or anionic residues of organic acids such as maleate, fumarate, oxalate, tartrate, citrate, lactate, or acetate.

A composition in which the amine and/or quaternized amine according to the general formulas (II) and/or (III) is an amidoamine and/or a quaternized amidoamine, in which R1 signifies a branched or unbranched, saturated or unsaturated acyl residue having 6 to 30 carbon atoms, which can include at least one OH group, is preferred. A fatty acid residue made of oils and waxes, in particular natural oils and waxes, is preferred here. Suitable examples thereof are lanolin, beeswax, or candelilla wax.

The alkylamidoamines are usually manufactured by amidation of natural or synthetic fatty acids and fatty acid cuts with dialkylaminoamines.

Also preferred are those amidoamines and/or quaternized amidoamines in which R², R³, and/or R⁴ in formulas (II) and/or (III) signify a residue according to the general formula CH₂CH₂OR⁵ in which R⁵ can have the meaning of alkyl residues having 1 to 4 carbon atoms, hydroxyethyl, or hydrogen. The preferred value of n in the general formulas (II) and/or (III) is an integer between 2 and 5.

Also preferred are amidoamines and/or quaternized amidoamines of the general formulas (II) and/or (III) in which the anion X⁻ is a halide ion or a compound of the general formula RSO₃⁻ in which R has the meaning of a saturated or unsaturated alkyl residue having 1 to 4 carbon atoms.

The alkyl residue having 1 to 4 carbon atoms of R², R³, and R⁴ and/or the alkyl residue having 1 to 4 carbon atoms of RSO₃⁻ in the general formula (II) and/or (III) can include at least one hydroxyl group.

The following amidoamines, for example, are appropriate as amidoamines to be used according to the present invention, which optionally can be quaternized: Witcamine 100 (Witco, INCI name: Cocamidopropyl Dimethylamine), Incromine BB (Croda, INCI name: Behenamidopropyl Dimethylamine), Mackine 401 (Mcintyre, INCI name: Isostearylamidopropyl Dimethylamine) and other Mackine grades, Adogen S18V (Witco, INCI name: Stearylamidopropyl Dimethylamine) and, as permanently cationic aminoamines: Rewoquat® 50 (Witco Surfactants GmbH, INCI name: Ricinoleamidopropyltrimonium Methosulfate), Empigen CSC (Albright & Wilson, INCI name: Cocamidopropyltrimonium Chloride), Swanol Lanoquat DES-50 (Nikko, INCI name: Quaternium-33), Rewoquat UTM 50 (Witco Surfactants GmbH, Undecyleneamidopropyltrimonium Methosulfate).

The amidoamines or quaternized amidoamines according to the general formulas (II) and (III) can be used individually or in any combinations with one another, quantities between 0.01 and 20 wt %, preferably quantities from 0.01 to 10 wt %, and very particularly preferably quantities from 0.1 to 7.5 wt % being included. The best results of all are obtained with quantities from 0.1 to 5 wt %, based in each case on the total composition of the respective agent.

Very particularly preferred hair treatment agents according to the present invention are characterized in that they include, based on their weight, 0.1 to 15 wt %, preferably 0.2 to 10 wt %, more preferably 0.3 to 7.5 wt %, even more preferably 0.4 to 5 wt %, and in particular 0.5 to 2.5 wt % dimethyl-N′-stearoyl-1,3-diaminopropane (INCI name: Stearamidopropyl Dimethylamine).

The agents according to the present invention include as ingredient c) at least one esterquat.

Esterquats are known substances. Such products are marketed, for example, under the trademarks Stepantex®, Dehyquart®, and Armocare®. The products Armocare® VGH-70—an N,N-bis(2-palmitoyloxyethyl)dimethylammonium chloride—as well as Dehyquart® F-75, Dehyquart® C-4046, Dehyquart® L80, and Dehyquart® AU-35 are examples of such esterquats.

Very particularly preferred hair treatment agents according to the present invention are characterized in that they include, based on their weight, 0.1 to 15 wt %, preferably 0.2 to 10 wt %, more preferably 0.3 to 7.5 wt %, even more preferably 0.4 to 5 wt %, and in particular 0.5 to 2.5 wt % distearoylethylhydroxyethylmethylammonium methosulfate (INCI name: Distearoylethyl Hydroxyethylmonium Methosulfate).

The effect of the combination of ingredients a), b), and c) is appreciably increased by the fact that the agents additionally include poly(2-methacryloxyethyltrimethylammonium chloride) (INCI name: Polyquaternium-37). The agents according to the present invention therefore include, as a further ingredient d), poly(2-methacryloxyethyltrimethylammonium chloride) (INCI name: Polyquaternium-37), a polymer of the general formula (IV)

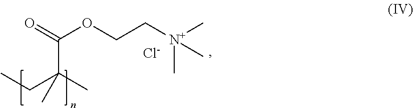

that is hydrogen-terminated at the ends.

Particularly preferred hair treatment agents according to the present invention include, based on their weight, 0.1 to 15 wt %, preferably 0.2 to 10 wt %, more preferably 0.3 to 7.5 wt %, even more preferably 0.4 to 5 wt %, and in particular 0.5 to 2.5 wt % poly(2-methacryloxyethyltrimethylammonium chloride) (INCI name: Polyquaternium-37).

Very particularly preferred agents according to the present invention include
a) 0.1 to 15 wt % of at least one quaternary imidazoline derivative of formula I in which R¹ and R² each denote —(CH₂)₂₀—CH₃ and X⁻ denotes CH₃—OSO₃⁻, and
b) 0.1 to 15 wt % dimethyl-N′-stearoyl-1,3-diaminopropane (INCI name: Stearamidopropyl Dimethylamine), and
c) 0.1 to 15 wt % distearoylethylhydroxyethylmethylammonium methosulfate (INCI name: Distearoylethyl Hydroxyethylmonium Methosulfate), and
d) 0.1 to 15 wt % poly(2-methacryloxyethyltrimethylammonium chloride) (INCI name: Polyquaternium-37).

Especially preferred hair treatment agents according to the present invention are characterized in that they include, based on their weight,
a) 0.5 to 2.5 wt % of at least one quaternary imidazoline derivative of formula I in which R¹ and R² each denote —(CH₂)₂₀—CH₃ and X⁻ denotes CH₃—OSO₃⁻,
b) 0.5 to 2.5 wt % dimethyl-N′-stearoyl-1,3-diaminopropane (INCI name: Stearamidopropyl Dimethylamine),
c) 0.5 to 2.5 wt % distearoylethylhydroxyethylmethylammonium methosulfate (INCI name: Distearoylethyl Hydroxyethylmonium Methosulfate),
d) 0.5 to 2.5 wt % poly(2-methacryloxyethyltrimethylammonium chloride) (INCI name: Polyquaternium-37).

The combination of four active substances included in the agents according to the present invention can be supplemented with further adjuvants. It has been found, for example, that esters of long-chain alcohols (fatty alcohols) with longer-chain monocarboxylic acids even further enhance the fullness and softness of the hair when they are incorporated into the agents according to the present invention. Hair treatment agents particularly preferred according to the present invention are characterized in that they additionally include esters of coconut fatty alcohols with octanoic acid.

Other emollients such as dicaprylyl carbonate, dicaprylyl ether, propylheptyl caprylate, etc. also exhibit these effects, but they are particularly pronounced with the coconut-alkyl esters of octanoic acid.

Very particularly preferred agents according to the present invention include
a) 0.1 to 15 wt % of at least one quaternary imidazoline derivative of formula I in which R¹ and R² each denote —(CH₂)₂₀—CH₃ and X⁻ denotes CH₃—OSO₃⁻, and
b) 0.1 to 15 wt % dimethyl-N′-stearoyl-1,3-diaminopropane (INCI name: Stearamidopropyl Dimethylamine), and
c) 0.1 to 15 wt % distearoylethylhydroxyethylmethylammonium methosulfate (INCI name: Distearoylethyl Hydroxyethylmonium Methosulfate), and
d) 0.1 to 15 wt % poly(2-methacryloxyethyltrimethylammonium chloride) (INCI name: Polyquaternium-37)
e) 0.1 to 10 wt % esters of coconut fatty alcohols with octanoic acid.

Especially preferred hair treatment agents according to the present invention are characterized in that they include, based on their weight,
a) 0.5 to 2.5 wt % of at least one quaternary imidazoline derivative of formula I in which R¹ and R² each denote —(CH₂)₂₀—CH₃ and X⁻ denotes CH₃—OSO₃⁻,
b) 0.5 to 2.5 wt % dimethyl-N′-stearoyl-1,3-diaminopropane (INCI name: Stearamidopropyl Dimethylamine),
c) 0.5 to 2.5 wt % distearoylethylhydroxyethylmethylammonium methosulfate (INCI name: Distearoylethyl Hydroxyethylmonium Methosulfate),
d) 0.5 to 2.5 wt % poly(2-methacryloxyethyltrimethylammonium chloride) (INCI name: Polyquaternium-37),
e) 0.1 to 10 wt % esters of coconut fatty alcohols with octanoic acid.

The combination of four (or, in the preferred embodiment, five) active substances included in the agents according to the present invention can be supplemented with further adjuvants. It has been found, for example, that while further specific QAVs and/or fatty alcohols cannot substantially further improve the care-providing effect, they can nevertheless appreciably extend it. Alkyltrimethylammonium halides and fatty alcohols have proven particularly suitable here; among these in turn, cetyltrimethylammonnium chloride and cetearyl alcohol are particularly preferred extenders of the care-providing effect.

Hair treatment agents particularly preferred according to the present invention are therefore characterized in that they include, based on their weight, 0.01 to 5 wt %, preferably 0.05 to 2.5 wt %, more preferably 0.1 to 2 wt %, even more preferably 0.15 to 1.5 wt %, and in particular 0.2 to 1 wt % cetyltrimethylammonium chloride.

Especially preferred hair treatment agents according to the present invention are characterized in that they include, based on their weight,
a) 0.5 to 2.5 wt % of at least one quaternary imidazoline derivative of formula I in which R¹ and R² each denote —(CH₂)₂₀—CH₃ and X⁻ denotes CH₃—OSO₃⁻,
b) 0.5 to 2.5 wt % dimethyl-N′-stearoyl-1,3-diaminopropane (INCI name: Stearamidopropyl Dimethylamine),
c) 0.5 to 2.5 wt % distearoylethylhydroxyethylmethylammonium methosulfate (INCI name: Distearoylethyl Hydroxyethylmonium Methosulfate),
d) 0.5 to 2.5 wt % poly(2-methacryloxyethyltrimethylammonium chloride) (INCI name: Polyquaternium-37),
e) 0.2 to 1 wt % cetyltrimethylammonium chloride.

Especially preferred hair treatment agents according to the present invention are characterized in that they include, based on their weight,
a) 0.5 to 2.5 wt % of at least one quaternary imidazoline derivative of formula I in which R¹ and R² each denote —(CH₂)₂₀—CH₃ and X⁻ denotes CH₃—OSO₃⁻,
b) 0.5 to 2.5 wt % dimethyl-N′-stearoyl-1,3-diaminopropane (INCI name: Stearamidopropyl Dimethylamine),
c) 0.5 to 2.5 wt % distearoylethylhydroxyethylmethylammonium methosulfate (INCI name: Distearoylethyl Hydroxyethylmonium Methosulfate),
d) 0.5 to 2.5 wt % poly(2-methacryloxyethyltrimethylammonium chloride) (INCI name: Polyquaternium-37),
e) 0.1 to 10 wt % esters of coconut fatty alcohols with octanoic acid,
f) 0.2 to 1 wt % cetyltrimethylammonium chloride.

Hair treatment agents likewise particularly preferred according to the present invention are therefore characterized in that they include, based on their weight, 0.01 to 5 wt %, preferably 0.05 to 2.5 wt %, more preferably 0.1 to 2 wt %, even more preferably 0.15 to 1.5 wt %, and in particular 0.2 to 1 wt % cetearyl alcohol.

It is particularly preferred to employ both cetylrimethylammonium chloride and cetearyl alcohol, since the care-providing effects of the agents according to the present invention are thereby supported and extended to a particular degree.

Very particularly preferred agents according to the present invention include
a) 0.1 to 15 wt % of at least one quaternary imidazoline derivative of formula I in which R¹ and R² each denote —(CH₂)₂₀—CH₃ and X⁻ denotes CH₃—OSO₃⁻, and
b) 0.1 to 15 wt % dimethyl-N′-stearoyl-1,3-diaminopropane (INCI name: Stearamidopropyl Dimethylamine), and
c) 0.1 to 15 wt % distearoylethylhydroxyethylmethylammonium methosulfate (INCI name: Distearoylethyl Hydroxyethylmonium Methosulfate), and
d) 0.1 to 15 wt % poly(2-methacryloxyethyltrimethylammonium chloride) (INCI name: Polyquaternium-37), and
e) 0.01 to 5 wt cetyltrimethylammonium chloride, and
f) 0.01 to 5 wt cetearyl alcohol.

Especially preferred hair treatment agents according to the present invention are characterized in that they include, based on their weight,
a) 0.5 to 2.5 wt % of at least one quaternary imidazoline derivative of formula I in which R¹ and R² each denote —(CH₂)₂₀—CH₃ and X⁻ denotes CH₃—OSO₃⁻,
b) 0.5 to 2.5 wt % dimethyl-N′-stearoyl-1,3-diaminopropane (INCI name: Stearamidopropyl Dimethylamine),
c) 0.5 to 2.5 wt % distearoylethylhydroxyethylmethylammonium methosulfate (INCI name: Distearoylethyl Hydroxyethylmonium Methosulfate),
d) 0.5 to 2.5 wt % poly(2-methacryloxyethyltrimethylammonium chloride) (INCI name: Polyquaternium-37),
e) 0.2 to 1 wt % cetyltrimethylammonium chloride
f) 0.2 to 1 wt % cetearyl alcohol.

Very particularly preferred agents according to the present invention include
a) 0.1 to 15 wt % of at least one quaternary imidazoline derivative of formula I in which R¹ and R² each denote —(CH₂)₂₀—CH₃ and X⁻ denotes CH₃—OSO₃⁻, and
b) 0.1 to 15 wt % dimethyl-N′-stearoyl-1,3-diaminopropane (INCI name: Stearamidopropyl Dimethylamine), and
c) 0.1 to 15 wt % distearoylethylhydroxyethylmethylammonium methosulfate (INCI name: Distearoylethyl Hydroxyethylmonium Methosulfate), and
d) 0.1 to 15 wt % poly(2-methacryloxyethyltrimethylammonium chloride) (INCI name: Polyquaternium-37), and
e) 0.1 to 10 wt % esters of coconut fatty alcohols with octanoic acid, and
f) 0.01 to 5 wt cetyltrimethylammonium chloride, and
g) 0.01 to 5 wt cetearyl alcohol.

The agents according to the present invention can include further ingredients.

With particular preference, the agents according to the present invention include amphoteric surfactant(s). “Amphoteric” surfactants and emulsifier agents are understood to be those surface-active compounds which include, in addition to a C₈ to C₂₄ alkyl or acyl group, at least one free amino group and at least one —COOH or —SO₃H group, and are capable of forming internal salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Particularly preferred ampholytic surfactants are N-cocalkylaminopropionate, cocacylaminoethylaminopropionate, and C₁₂ to C₁₈ acyl sarcosine.

Preferred hair treatment agents according to the present invention are characterized in that they include 1 to 30 wt %, preferably 6 to 25 wt %, more preferably 7 to 20 wt %, even more preferably 8 to 15 wt %, and in particular 10 to 12.5 wt % amphoteric surfactant(s).

Particularly preferred hair treatment agents according to the present invention are characterized in that they include amphoteric surfactant(s) from the groups of
N-alkylglycines,
N-alkylpropionic acids,
N-alkylaminobutyric acids,
N-alkyliminodipropionic acids,
N-hydroxyethyl-N-alkylamidopropyl glycines,
N-alkyltaurines,
N-alkylsarcosines,
2-alkylaminopropionic acids each having approximately 8 to 24 carbon atoms in the alkyl group,
alkylaminoacetic acids each having approximately 8 to 24 carbon atoms in the alkyl group,
N-cocalkylaminopropionate,
cocacylaminoethylaminopropionate,
C₁₂ to C₁₈ acyl sarcosine,
N-alkyl-N,N-dimethylammonium glycinates, for example cocalkyldimethylammonium glycinate,
N-acylaminopropyl-N,N-dimethylammonium glycinates, for example cocacylaminopropyldimethylammonium glycinate,
2-alkyl-3-carboxymethyl-3-hydroxyethylimidazolines each having 8 to 18 carbon atoms in the alkyl or acyl group,
cocacylaminoethylhydroxyethylcarboxymethyl glycinate,
the compounds known by the INCI name Cocamidopropyl Betaine,
the compounds known by the INCI name Disodium Cocamphodiacetate,
   wherein preferred agents include the amphoteric surfactant(s) in quantities from 0.5 to 9 wt %, preferably from 0.75 to 8 wt %, and in particular from 1 to 7.5 wt %, based in each case on the total agent.

Particularly preferred hair treatment agents include, as amphoteric surfactants, betaines of formula (Bet-I)

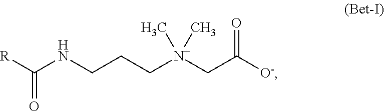

in which R denotes a straight-chain or branched, saturated or mono- or polyunsaturated alkyl or alkenyl residue having 8 to 24 carbon atoms.

These surfactants are referred to according to INCI nomenclature as Amidopropyl Betaines, the representatives that derive from coconut fatty acids being preferred, and being referred to as Cocamidopropyl Betaines. It is particularly preferred according to the present invention to use surfactants of formula (Bet-I) that are a mixture of the following representatives:

H₃C—(CH₂)₇—C(O)—NH—(CH₂)₃N⁺(CH₃)₂CH₂COO⁻

H₃C—(CH₂)₉—C(O)—NH—(CH₂)₃N⁺(CH₃)₂CH₂COO⁻

H₃C—(CH₂)₁₁—C(O)—NH—(CH₂)₃N(CH₃)₂CH₂COO⁻

H₃C—(CH₂)₁₃—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₇—CH═CH—(CH₂)₇—C(O)—NH—(CH₂)₃N⁺(CH₃)₂CH₂COO⁻.

Particularly preferably, surfactants of formula (Bet-I) are used within narrower quantity ranges. Agents according to the present invention that include, based on their weight, 0.25 to 8 wt %, more preferably 0.5 to 7 wt %, more preferably 0.75 to 6.5 wt %, and in particular 1 to 5.5 wt % surfactant(s) of formula (Bet-1) are preferred here.

In addition to or instead of the amphoteric surfactant(s) of formula (Bet-I), the hair treatment agents according to the present invention can with particular preference include, as amphoteric surfactants, betaines of formula (Bet-II)

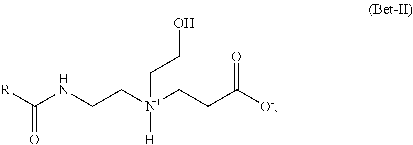

in which R denotes a straight-chain or branched, saturated or mono- or polyunsaturated alkyl or alkenyl residue having 8 to 24 carbon atoms.

These surfactants are referred to according to INCI nomenclature as Amphoacetates, the representatives derived from coconut fatty acids being preferred and being referred to as Cocoamphoacetates.

For production-engineering reasons, surfactants of this type also always include betaines of formula (Bet-IIa)

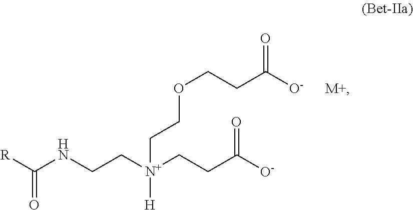

in which R denotes a straight-chain or branched, saturated or mono- or polyunsaturated alkyl or alkenyl residue having 8 to 24 carbon atoms, and M denotes a cation.

These surfactants are referred to according to INCI nomenclature as Amphodiacetates, the representatives derived from coconut fatty acids being preferred and being referred to as Cocoamphodiacetates.

It is particularly preferred to use according to the present invention surfactants of formula (Bet-II) that are a mixture of the following representatives:

H₃C—(CH₂)₇—C(O)—NH—(CH₂)₂NH⁺(CH₂CH₂OH)CH₂CH₂COO⁻,

H₃C—(CH₂)₉—C(O)—NH—(CH₂)₂NH⁺(CH₂CH₂OH)CH₂CH₂COO⁻,

H₃C—(CH₂)₁₁—C(O)—NH—(CH₂)₂NH⁺(CH₂CH₂OH)CH₂CH₂COO⁻,

H₃C—(CH₂)₁₃—C(O)—NH—(CH₂)₂NH⁺(CH₂CH₂OH)CH₂CH₂COO⁻,

H₃C—(CH₂)₁₅—C(O)—NH—(CH₂)₂NH⁺(CH₂CH₂OH)CH₂CH₂COO⁻,

H₃C—(CH₂)₇—CH═CH—(CH₂)₇—C(O)—NH—(CH₂)₂NH⁺(CH₂CH₂₀H)CH₂CH₂COO⁻.

Surfactants of formula (Bet-II) are used particularly preferably within narrower quantity ranges. Agents according to the present invention that include, based on their weight, 0.25 to 8 wt %, more preferably 0.5 to 7 wt %, more preferably 0.75 to 6.5 wt %, and in particular 1 to 5.5 wt % surfactant(s) of formula (Bet-II) are preferred here.

In addition to or instead of the amphoteric surfactants, the agents according to the present invention can also include nonionic surfactants and/or cationic surfactants (see above).

Particularly preferred nonionic surfactants are alkylpolyglycosides. Alkylpolyglycosides (APG) are nonionic surfactants that are manufactured entirely from renewable raw materials (sugar modules, principally glucose, for example from corn starch, and fatty alcohol, for example from coconut oil). Alkylpolyglycosides are accessible via an acid-catalyzed reaction (Fischer reaction) from sugars, in particular glucose (or starch) or from butylglycosides with fatty alcohols.

This results in complex mixtures of alkylmonoglucoside (alkyl-α-D- and -β-D-glucopyranoside as well as small proportions of -glucofuranoside), alkyldiglucosides (-isomaltosides, -maltosides, etc.), and alkyloligoglucosides (-maltotriosides, -tetraosides, etc.). The average degree of polymerization of commercial products, whose alkyl residues are in the range from C8 to C16, is 1.2 to 1.5.

It is preferred according to the present invention to use alkylpolyglycosides corresponding to the general formula RO-(Z)ₓ, where R denotes alkyl, Z denotes sugar, and x denotes the number of sugar units.

Those alkylpolyglycosides in which R is made up
substantially of C₈ and C₁₀ alkyl groups,
substantially of C₁₂ and C₁₄ alkyl groups,
substantially of C₈ to C₁₆ alkyl groups, or
substantially of C₁₂ to C₁₆ alkyl groups, or
substantially of C₁₆ to C₁₈ alkyl groups
   are particularly preferred.

Any mono- or oligosaccharides can be used as sugar module Z. Sugars having 5 or 6 carbon atoms, as well as the corresponding oligosaccharides, are usually used. Such sugars are, for example, glucose, fructose, galactose, arabinose, ribose, xylose, lyxose, allose, altrose, mannose, gulose, idose, talose, and sucrose. Preferred sugar modules are glucose, fructose, galactose, arabinose, and sucrose.

Preferred hair treatment agents according to the present invention are therefore characterized in that they include, based on their weight, 0.1 to 20 wt %, preferably 1 to 10 wt %, and in particular 2 to 8 wt % alkylpolyglycoside(s) of the formula

H₃C—(CH₂)ₙ—O—(Z)ₓ,

in which n denotes values from 5 to 21, preferably from 7 to 19, particularly preferably from 9 to 17, and in particular from 11 to 13, and k denotes values from 1.1 to 1.8, preferably from 1.2 to 1.5, and Z denotes a sugar module from the group of glucose, fructose, galactose, arabinose, ribose, xylose, lyxose, allose, altrose, mannose, gulose, idose, talose, and sucrose.

Glucose is a particularly preferred sugar module (Z), so that preferred hair treatment agents according to the present invention are characterized in that they include, based on their weight, 0.1 to 15 wt %, preferably 1 to 10 wt %, and in particular 2 to 8 wt % alkylpolyglucoside(s) of the formula

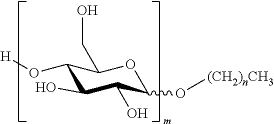

in which n denotes values from 5 to 21, preferably from 7 to 19, particularly preferably from 9 to 17, and in particular from 11 to 13, and m denotes values from 1.1 to 1.8, preferably from 1.2 to 1.5.

The alkylpolyglycosides usable according to the present invention include on average 1.1 to 5 sugar units. Alkylpolyglycosides having values of x from 1.1 to 2.0 are preferred. Alkylglycosides in which x is equal to 1.1 to 1.8 are very particularly preferred.

Very particularly preferred alkylpolyglucosides are those whose alkyl residue is a lauryl residue. In the context of substance mixtures from natural sources, those sources which have a high proportion of C12 fatty acids, in particular coconut fatty acids, are preferred.

The care-providing effects of the agents according to the present invention can be further intensified by employing specific care-providing substances. The latter are preferably selected from specific groups of care-providing substances known per se, since these care-providing substances harmonize outstandingly with the combination according to the present invention in terms of formulation engineering and care-providing effect.

Hair treatment agents preferred according to the present invention are characterized in that they additionally include care-providing substance(s), based on their weight, in quantities from 0.001 to 10 wt %, preferably 0.005 to 7.5 wt %, particularly preferably 0.01 to 5 wt %, and in particular 0.05 to 2.5 wt %, wherein preferred care-providing substance(s) are selected from the group
i. L-carnitine and/or salts thereof;
ii. taurine and/or salts thereof;
iii. niacinamide;
iv. ubiquinone;
v. ectoin.

L-carnitine (IUPAC name: (R)-(3-carboxy-2-hydroxypropyl)-N,N,N-trimethylammonium hydroxide) is a naturally occurring vitamin-like substance.

As a betaine, L-carnitine can form addition compounds and double salts. L-carnitine derivatives preferred according to the present invention are selected in particular from acetyl-L-carnitine, L-carnitine fumarate, L-carnitine citrate, lauroyl-L-carnitine, and particularly preferably L-carnitine tartrate. The aforesaid L-carnitine compounds are obtainable, for example, from Lonza GmbH (Wuppertal, Germany).

Preferred hair treatment agents according to the present invention are characterized in that they include, based on their weight, 0.001 to 10 wt %, preferably 0.005 to 7.5 wt %, particularly preferably 0.01 to 5 wt %, and in particular 0.05 to 2.5 wt % L-carnitine or L-carnitine derivatives, preferred L-carnitine derivatives being selected from acetyl-L-carnitine, L-carnitine fumarate, L-carnitine citrate, lauroyl-L-carnitine, and in particular L-carnitine tartrate.

A further care-providing substance that is preferred for use, which possesses activating properties, is taurine. Hair treatment agents preferred according to the present invention include, based on their weight, 0.01 to 15 wt %, preferably 0.025 to 12.5 wt %, particularly preferably 0.05 to 10 wt %, more preferably 0.1 to 7.5 wt %, and in particular 0.5 to 5 wt % taurine (2-aminoethanesulfonic acid).

A further preferred group of care-providing substances in the agents according to the present invention is vitamins, provitamins, or vitamin precursors. These are described below:

The group of substances referred to as “vitamin A” includes retinol (vitamin A₁) as well as 3,4-didehydroretinol (vitamin A₂). β-Carotene is the provitamin of retinol. Vitamin A components that are suitable according to the present invention are, for example, vitamin A acid and esters thereof, vitamin A aldehyde, and vitamin A alcohol, as well as esters thereof such as the palmitate and the acetate. The agents according to the present invention include the vitamin A component preferably in quantities from 0.05 to 1 wt %, based on the total preparation.

Members of the vitamin B group or vitamin B complex are, among others:
Vitamin B₁ (thiamine)
Vitamin B₂ (riboflavin)
Vitamin B₃. The compounds nicotinic acid and nicotinic acid amide (niacinamide) are often listed under this designation. Nicotinic acid amide is preferred according to the present invention; it is included in the agents used according to the present invention preferably in quantities from 0.05 to 1 wt % based on the total agent.
Vitamin B₅ (pantothenic acid, panthenol, and pantolactone). In the context of this group, panthenol and/or pantolactone are preferably used (see below). Derivatives of panthenol that are usable according to the present invention are, in particular, the esters and ethers of panthenol as well as cationically derivatized panthenols. Individual representatives are, for example, panthenol triacetate, panthenol monoethyl ether and the monoacetate thereof, as well as cationic panthenol derivatives disclosed in WO 92/13829. The aforesaid compounds of the vitamin B₅ type are included in the agents according to the present invention preferably in quantities from 0.05 to 10 wt % based on the total agent. Quantities from 0.1 to 5 wt % are particularly preferred.
Vitamin B₆ (pyridoxine as well as pyridoxamine and pyridoxal).

Vitamin C (ascorbic acid). Vitamin C is utilized in the agents according to the present invention preferably in quantities from 0.1 to 3 wt % based on the total agent. Utilization in the form of the palmitic acid ester, glucosides, or phosphates can be preferred. Utilization in combination with tocopherols can likewise be preferred.

Vitamin E (tocopherols, in particular c-tocopherol). Tocopherol and its derivatives, which include in particular esters such as the acetate, nicotinate, phosphate, and succinate, are included in the agents according to the present invention preferably in quantities from 0.05 to 1 wt % based on the total agent.

Vitamin F. The term “vitamin F” is usually understood to mean essential fatty acids, in particular linoleic acid, linolenic acid, and arachidonic acid.

Vitamin H. “Vitamin H” refers to the compound (3aS,4S,6aR)-2-oxohexahydrothienol[3,4-d]-imidazole-4-valeric acid, for which the trivial name “biotin” has, however, now become established. Biotin is included in the agents according to the present invention preferably in quantities from 0.0001 to 1.0 wt %, in particular in quantities from 0.001 to 0.01 wt %.

In summary, hair treatment agents according to the present invention that include, based on their weight, 0.1 to 5 wt %, preferably 0.2 to 4 wt %, particularly preferably 0.25 to 3.5 wt %, more preferably 0.5 to 3 wt %, and in particular 0.5 to 2.5 wt % vitamins and/or provitamins and/or vitamin precursors that preferably are assigned to the groups A, B, C, E, F, and H, wherein preferred agents include-2,4-dihydroxy-N-(3-hydroxypropyl)-3,3-dimethylbutyramide (provitamin B₅) and/or pantothenic acid (vitamin B₃, vitamin Bs) and/or niacin, niacinamide or nicotinamide (vitamin B₃) and/or L-ascorbic acid (vitamin C) and/or thiamin (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂, are preferred.

It has been found that specific quinones have a particular suitability as a care-providing substance. The agents according to the present invention can therefore include, as a further care-providing substance, 0.0001 to 5 wt % of at least one bioquinone of formula (Ubi)

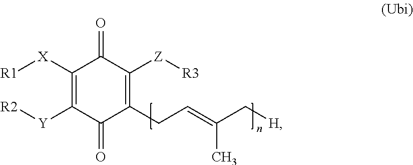

in which
X, Y, Z mutually independently denote —O— or —NH— or —NR⁴— or a chemical bond, R¹, R², R³ mutually independently denote a hydrogen atom or an optionally substituted aryl group or an optionally substituted (C₁ to C₆) alkyl group or a hydroxyalkyl group or a polyhydroxyalkyl group or an optionally substituted (C₁ to C₆) alkylene group or a (C₁ to C₆) acyl group, wherein preferred residues are selected mutually independently from —H, —CH₃, —CH₂CH₃, —(CH₂)₂CH₂, —CH(CH₃)₂, —CH₂)₃CH₃, —CH(CH₃)CH₂CH₃, —CH₂CH(CH₃)₂, —C(CH₃)₃,
R⁴ denotes —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃,
n denotes values from 1 to 20, preferably from 2 to 15, and in particular 5, 6, 7, 8, 9, 10.

Particularly preferred hair treatment agents according to the present invention are characterized in that they include as a care-providing substance, based on their weight, 0.0001 to 1 wt %, preferably 0.001 to 0.5 wt %, and particularly preferably 0.005 to 0.1 wt % of at least one ubiquinone and/or at least one ubiquinol and/or at least one derivative of said substances, wherein preferred agents include a ubiquinone of formula (Ubi)

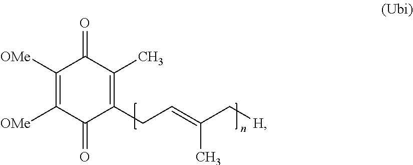

in which n denotes the values 6, 7, 8, 9, or 10, particularly preferably 10 (Coenzyme Q10).

Alternatively or in addition to the particularly preferred ubiquinones, the agents according to the present invention can also include plastoquinones. Preferred agents according to the present invention are characterized here in that they include 0.0002 to 4 wt %, preferably 0.0005 to 3 wt %, particularly preferably 0.001 to 2 wt %, more preferably 0.0015 to 1, and in particular 0.002 to 0.5 wt % of at least one plastoquinone of formula (Ubi-b)

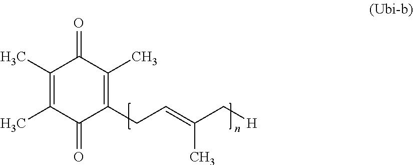

in which n denotes values from 1 to 20, preferably from 2 to 15, and in particular 5, 6, 7, 8, 9, 10.

As a further care enhancer, the agents according to the present invention can include ectoin. Ectoin ((4S)-2-methyl-1,4,5,6-tetrahydropyrimidine-4-carboxylic acid) is a natural substance belonging to the group of the compatible solutes. This highly water-bonding low-molecular-weight organic compound occurs in halophilic bacteria and allows these extremophile organisms to survive under stress conditions. Hair treatment agents preferred according to the present invention are characterized in that they include, based on their weight, 0.001 to 10 wt %, preferably 0.01 to 5 wt %, particularly preferably 0.05 to 2.5 wt %, and in particular 0.1 to 1 wt % (S)-2-methyl-1,4,5,6-tetrahydro-4-pyrimidinecarboxylic acid (ectoin) as well as the physiologically acceptable salts of that compound, and/or (S,S)-5-hydroxy-2-methyl-1,4,5,6-tetrahydro-4-pyrimidinecarboxylic acid (hydroxyectoin) as well as the physiologically acceptable salts of that compound.

To improve the elasticity and strengthen the internal structure of hair treated with the agents according to the present invention, the agents according to the present invention can include purine and/or purine derivatives as a care-providing substance. In particular, the result of combining purine and/or purine derivatives with ubiquinones and/or plastoquinones as a care-providing substance is that hair treated with corresponding agents exhibits, inter alia, higher measured values in differential thermal analysis and improved wet and dry combability values.

Purine (7H-imidazo[4,5-d]pyrimidine) does not occur in isolation in nature, but constitutes the basic member of the purines. Purines in turn are a group of important compounds, widespread in nature and involved in human, animal, plant, and microbial metabolic processes, that derive from the basic member by substitution with OH, NH₂, SH in the 2-, 6-, and 8-position, and/or with CH₃ in the 1-, 3-, 7-position. Purine can be manufactured, for example, from aminoacetonitrile and formamide. Purines and purine derivatives are often isolated as natural substances, but are also accessible synthetically by numerous routes.

Preferred agents according to the present invention include purine and/or purine derivatives in narrower quantity ranges. Cosmetic agents preferred according to the present invention are characterized here in that they include, based on their weight, 0.001 to 2.5 wt %, preferably 0.0025 to 1 wt %, particularly preferably 0.005 to 0.5 wt %, and in particular 0.01 to 0.1 wt % purine(s) and/or purine derivative(s).

Among purine, the purines, and the purine derivatives, some representatives are particularly preferred according to the present invention. Hair treatment agents preferred according to the present invention are characterized in that they include as a care-providing substance, based on their weight, 0.001 to 2.5 wt %, preferably 0.0025 to 1 wt %, particularly preferably 0.005 to 0.5 wt %, and in particular 0.01 to 0.1 wt % purine(s) and/or purine derivative(s), wherein preferred agents include purine and/or purine derivative(s) of formula (Pur-I)

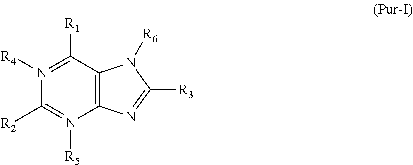

in which the residues R¹, R², and R³ are selected mutually independently from —H, —OH, NH₂, —SH, and the residues R⁴, R⁵, and R⁶ are selected mutually independently from —H, —CH₃, and —CH₂—CH₃, the following compounds being preferred:
purine (R¹═R²=R³═R⁴=R⁵═R⁶═H), adenine (R¹═NH₂, R²═R³=R⁴═R⁵═R⁶═H), guanine (R¹═OH, R²═NH₂, R³═R⁴=R⁵═R⁶=H), uric acid (R¹═R²=R³═OH, R⁴═R⁵=R⁶═H), hypoxanthine (R¹═OH, R²═R³=R⁴═R⁵=R⁶═H), 6-purinethiol (R¹═SH, R²═R³=R⁴═R⁵=R⁶═H), 6-thioguanine (R¹═SH, R²═NH₂, R³═R⁴=R⁵═R⁶=H), xanthine (R¹═R²═OH, R³═R⁴=R⁵═R⁶=H), caffeine (R¹═R²═OH, R³═H, R⁴═R⁵=R⁶═CH₃), theobromine (R¹═R²═OH, R³═R⁴=H, R⁵═R⁶=CH₃), theophylline (R¹═R²═OH, R³═H, R⁴═CH₃, R⁵═CH₃, R⁶═H).

It is further advantageous to use purine or purine derivatives and bioquinones at a specific ratio to one another. Preferred in this context are agents according to the present invention in which the weight ratio of purine (derivative(s)) to bioquinone(s) is equal to 10:1 to 1:100, preferably 5:1 to 1:50, particularly preferably 2:1 to 1:20, and in particular 1:1 to 1:10.

As already mentioned, caffeine is a particularly preferred purine derivative and Coenzyme Q10 is a particularly preferred bioquinone. Particularly preferred agents according to the present invention are therefore characterized in that they include, based on their weight, 0.001 to 2.5 wt %, preferably 0.0025 to 1 wt %, particularly preferably 0.005 to 0.5 wt %, and in particular 0.01 to 0.1 wt % caffeine, and 0.0002 to 4 wt %, preferably 0.0005 to 3 wt %, particularly preferably 0.001 to 2 wt %, more preferably 0.0015 to 1, and in particular 0.002 to 0.5 wt % Coenzyme Q10.

The agents according to the present invention can also include flavonoids as a care-providing substance. Flavonoids are a group of water-soluble vegetable dyes and play an important role in the metabolism of many plants. Together with the phenolic acids, they belong to the polyphenols. Well over 6,500 different flavonoids are known, and can be subdivided into flavonols, flavones, flavanones, isoflavanoids, and anthocyans.

Flavonoids from all six groups can be used according to the present invention, specific representatives from the individual groups being preferred as a care-providing substance because of their particularly intense effect. Preferred flavonols are quercetin, rutin, camphor oil, myricetin, isorhamnetin, preferred flavanols are catechin, gallocatechin, epicatechin, epigallocatechin gallate, theaflavin, thearubigin, preferred flavones are luteolin, apigenin, morin, preferred flavanones are hesperetin, naringenin, eriodictyol, preferred isoflavanoids are genistein, daidzein, and preferred anthocyanidins (anthocyans) are cyanidin, delphinidin, malvidin, pelargonidin, peonidin, petunidin.

Hair treatment agents particularly preferred according to the present invention are characterized in that they include, based on their weight, 0.001 to 2.5 wt %, preferably 0.0025 to 1 wt %, particularly preferably 0.005 to 0.5 wt %, and in particular 0.01 to 0.1 wt % flavonoids, in particular flavonols, particularly preferably 3,3′,4′,5,7-pentahydroxyflavone (quercetin) and/or 3,3′,4′,5,7-pentahydroxyflavone-3-O-rutinoside (rutin).

The use of bisabolol and/or bisabolol oxides as a care-providing agent in the agents according to the present invention is also preferred. Hair treatment agents according to the present invention that additionally include 0.001 to 5 wt %, preferably 0.01 to 4 wt %, particularly preferably 0.02 to 2.5 wt %, and in particular 0.1 to 1.5 wt % bisabolol and/or oxides of bisabolol, preferably (−)-alpha-bisabolol,

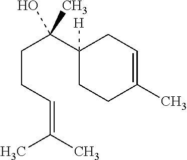 [SMILES: CC(C)=CCC[C@](C)(O)[C@H]1CC=C(C)CC1]

are preferred here.

Creatine is also suitable according to the present invention as a care-providing substance. Creatine (3-methylguanidinoacetic acid) is an organic acid that in vertebrates, inter alia, contributes to supplying muscles with energy. Creatine is synthesized in the kidneys, the liver, and the pancreas. It is derived formally from the amino acids glycine and arginine, and 95% of it is present in skeletal muscle.

Particularly preferred hair treatment agents according to the present invention include, based on their weight, 0.01 to 15 wt %, preferably 0.025 to 12.5 wt %, particularly preferably 0.05 to 10 wt %, more preferably 0.1 to 7.5 wt %, and in particular 0.5 to 5 wt % N-methylguanidinoacetic acid (creatine).

The agents according to the present invention can include, in addition to the ingredients recited above and optional further ingredients, further substances that prevent, mitigate, or cure hair loss. A content of hair-root-stabilizing active agents is particularly advantageous. These substances are described below:

Propecia (finasteride) is at present the only preparation that is approved worldwide and for which effectiveness and compatibility have been demonstrated in numerous studies. The effect of Propecia is that less DHT can form from testosterone.

Minoxidil, with or without supplementary additives, is probably the oldest demonstrably effective hair growth agent. For the treatment of hair loss, it must only be used for external application. Hair lotions exist that include 2% to 5% minoxidil, also gels having up to 15% minoxidil. Effectiveness increases with dosage, but minoxidil is soluble in hair lotions only up to a 5% proportion. In many countries, hair lotions having a minoxidil content of up to 2% are obtainable without a prescription.

To counteract hormonal influences on the hair follicles, spironolactone can be applied for external use in the form of a hair lotion and in combination with minoxidil. Spironolactone acts as an androgen receptor blocker, i.e. the binding of DHT to the hair follicles is prevented.

In summary, hair treatment agents according to the present invention that additionally include, based on its weight, 0.001 to 5 wt % hair-root-stabilizing substances, in particular minoxidil and/or finasteride and/or ketoconazole, are preferred.

In addition to the care-providing substances, the agents according to the present invention can include further care-providing substances. Their presence is not obligatorily necessary in order for the effects according to the present invention to be achieved, but farther-reaching effects, such as a pleasant feel or pleasant application haptics, may result from the use of these care-providing substances.

The agents according to the present invention can, with particular preference, include one or more amino acids as a further ingredient. Amino acids usable particularly preferably according to the present invention derive from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the present invention include one or more amino acids in narrower quantity ranges. Hair treatment agents preferred according to the present invention are characterized here in that they include as a care-providing substance, based on their weight, 0.01 to 5 wt %, preferably 0.02 to 2.5 wt %, particularly preferably 0.05 to 1.5 wt %, more preferably 0.075 to 1 wt %, and in particular 0.1 to 0.25 wt % amino acid(s), preferably from the group of glycine and/or alanine and/or valine and/or lysine and/or leucine and/or threonine.

As a further constituent, the agents according to the present invention can include at least one carbohydrate from the group of the monosaccharides, disaccharides, and/or oligosaccharides. Hair treatment agents preferred according to the present invention are characterized here in that they include as a care-providing substance, based on their weight, 0.01 to 5 wt %, preferably 0.05 to 4.5 wt %, particularly preferably 0.1 to 4 wt %, more preferably 0.5 to 3.5 wt %, and in particular 0.75 to 2.5 wt % carbohydrate(s) selected from monosaccharides, disaccharides, and/or oligosaccharides, preferred carbohydrates being selected from
monosaccharides, in particular D-ribose and/or D-xylose and/or L-arabinose and/or D-glucose and/or D-mannose and/or D-galactose and/or D-fructose and/or sorbose and/or L-fucose and/or L-rhamnose,
disaccharides, in particular sucrose and/or maltose and/or lactose and/or trehalose and/or cellobiose and/or gentobiose and/or isomaltose.

Particularly preferred agents according to the present invention include, based on their weight,
0.005 to 0.015 wt % caffeine and 0.75 to 1.5 wt % glucose monohydrate,
0.005 to 0.015 wt % caffeine and 0.75 to 1.5 wt % sucrose,
0.005 to 0.015 wt % caffeine and 0.75 to 1.5 wt % fructose.

As already mentioned, preferred agents according to the present invention include (an) amino acids(s).

Amino acids usable particularly preferably according to the present invention derive from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cyss), L-carnitine, L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the present invention include one or more amino acids in narrower quantity ranges. Cosmetic agents preferred according to the present invention are characterized here in that they additionally include 0.05 to 5 wt %, preferably 0.1 to 2.5 wt %, particularly preferably 0.15 to 1 wt %, and in particular 0.2 to 0.5 wt % amino acid(s), preferably (an) amino acid(s) from the group of glycine and/or alanine and/or valine and/or lysine and/or leucine and/or threonine.

Silicones represent a particularly preferred group of ingredients.

Agents preferred according to the present invention are characterized in that they include at least one silicone, preferably a silicone that is selected from among:
(i) polyalkylsiloxanes, polyarylsiloxanes, polyalkylarylsiloxanes, which are volatile or nonvolatile, straight-chain, branched, or cyclic, crosslinked or not crosslinked;
(ii) polysiloxanes that include in their general structure one or more organofunctional groups that are selected from among:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   ) sulfonic acid groups; and
   m) sulfate or thiosulfate groups;
(iii) linear polysiloxane (A)-polyoxyalkylene (B) block copolymers of the (A-B)ₙ type, where n>3;
(iv) grafted silicone polymers having a non-silicone-including organic backbone that is made up of an organic main chain that is formed from organic monomers that include no silicone, onto which, in the chain and optionally at at least one end of the chain, at least one polysiloxane macromer has been grafted;
(v) grafted silicone polymers having a polysiloxane backbone onto which have been grafted non-silicone-including organic monomers that have a polysiloxane main chain onto which, in the chain and optionally at at least one of its ends, at least one organic macromer that includes no silicone has been grafted; or mixtures thereof.

Agents particularly preferred according to the present invention include silicone(s) preferably in quantities from 0.1 to 10 wt %, preferably from 0.25 to 7 wt %, and in particular from 0.5 to 5 wt %, based in each case on the total agent.

Preferred silicones are described below.

Particularly preferred agents according to the present invention are characterized in that they include at least one silicone of formula (Si1.2)

(CH₃)₃Si—[O—Si(CH₃)₂]ₓ—O—Si(CH₃)₃ (Si-I),

in which x denotes a number from 0 to 100, preferably from 0 to 50, more preferably from 0 to 20, and in particular 0 to 10.

These silicones are referred to according to INCI nomenclature as Dimethicones.

Preferred silicones usable according to the present invention have viscosities from 0.2 to 2 mm² s⁻¹ at 20° C., silicones having viscosities from 0.5 to 1 mm² s⁻¹ being particularly preferred.

Particularly preferred agents according to the present invention include one or more aminofunctional silicones. Such silicones can be described, for example, by the formula

M(RₐQ_{b}SiO_{(4-a-b)/2)x}(R_{c}SiO_{(4-c)/2)y}M;

in the above formula, R is a hydrocarbon or hydrocarbon residue having 1 to approximately 6 carbon atoms, Q is a polar residue of the general formula —R¹HZ in which R¹ is a divalent connecting group that is bound to hydrogen and to the Z residue, assembled from carbon and hydrogen atoms, carbon, hydrogen, and oxygen atoms, or carbon, hydrogen, and nitrogen atoms, and Z is an organic aminofunctional residue that includes at least one aminofunctional group; “a” assumes values in the range from approximately 0 to approximately 2, “b” assumes values in the range from approximately 1 to approximately 3, “a”+“b” is less than or equal to 3, and “c” is a number in the range from approximately 1 to approximately 3, and x is a number in the range from 1 to approximately 2,000, preferably from approximately 3 to approximately 50, and most preferably from approximately 3 to approximately 25, and y is a number in the range from approximately 20 to approximately 10,000, preferably from approximately 125 to approximately 10,000, and most preferably from approximately 150 to approximately 1,000, and M is a suitable silicone terminal group that is known in the existing art, preferably trimethylsiloxy. Non-limiting examples of the residues represented by R include alkyl residues such as methyl, ethyl, propyl, isopropyl, isopropyl, butyl, isobutyl, amyl, isoamyl, hexyl, isohexyl and the like; alkenyl residues such as vinyl, halovinyl, alkylvinyl, allyl, haloallyl, alkylallyl; cycloalkyl residues such as cyclobutyl, cyclopentyl, cyclohexyl and the like; phenyl residues, benzyl residues, halogenated hydrocarbon residues such as 3-chloropropyl, 4-bromobutyl, 3,3,3-trifluoropropyl, chlorocyclohexyl, bromophenyl, chlorophenyl, and the like, and sulfur-including residues such as mercaptoethyl, mercaptopropyl, mercaptohexyl, mercaptophenyl and the like; R is preferably an alkyl residue that includes 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂, —CH₂CH(CH₃)C(O)OCH₂—, —(CH₂)₃C(O)OCH₂CH₂—, —C₆H₄C₆H₄—, —C₆H₄CH₂C₆H₄—, and —(CH₂)₃C(O)SCH₂CH₂—.

Z is an organic aminofunctional residue including at least one functional amino group. One possible formula for Z is NH(CH₂)_{z}NH₂, in which z is 1 or more. Another possible formula for Z is —NH(CH₂)_{z}NH(CH₂)_{zz}, in which both z and zz are mutually independently 1 or more; this structure comprises diamino ring structures such as piperazinyl. Z is most preferably a —NHCH₂CH₂NH₂ residue. Another possible formula for Z is —N(CH₂)_{z}(CH₂)_{zz}NX₂ or —NX₂, in which each X is selected, independently of X₂, from the group consisting of hydrogen and alkyl groups having 1 to 12 carbon atoms, and zz is 0.

Q is most preferably a polar aminofunctional residue of the formula —CH₂CH₂CH₂NHCH₂CH₂NH₂. In the formulas, “a” assumes values in the range from approximately 0 to approximately 2, “b” assumes values in the range from approximately 2 to approximately 3, “a”+“b” is less than or equal to 3, and “c” is a number in the range from approximately 1 to approximately 3. The molar ratio of the RₐQ_{b}SiO_{(4-a-b)/2} units to the R_{c}SiO_{(4-c)/2} units is in the range from approximately 1:2 to 1:65, preferably from approximately 1:5 to approximately 1:65, and most preferably from approximately 1:15 to approximately 1:20. If one or more silicones of the above formula are used, the different variable substituents in the above formula can then be different in the different silicone components that are present in the silicone mixture.

Particularly preferred agents according to the present invention are characterized in that they include at least one aminofunctional silicone of formula (Si-IIa)

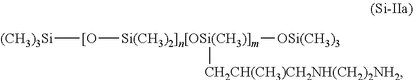

in which m and n are numbers whose sum (m+n) is between 1 and 2000, preferably between 50 and 150, wherein n assumes values preferably from 0 to 1999 and in particular from 49 to 149, and m assumes values preferably from 1 to 2000, in particular from 1 to 10.

These silicones are referred to according to the INCI declaration as Trimethylsilylamodimethicones.

Also particularly preferred are agents according to the present invention that include an aminofunctional silicone of formula (Si-IIb)

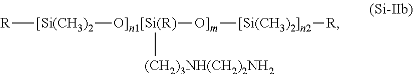

in which R denotes —OH, —O—CH₃, or a —CH₃ group, and m, n1, and n2 are numbers whose sum (m+n1+n2) is between 1 and 2000, preferably between 50 and 150, where the sum (n1+n2) assumes values preferably from 0 to 1999 and in particular from 49 to 149, and m assumes values preferably from 1 to 2000, in particular from 1 to 10.

These silicones are referred to according to the INCI declaration as Amodimethicones.

Regardless of which aminofunctional silicones are used, agents according to the present invention that include an aminofunctional silicone whose amine number is above 0.25 meq/g, preferably above 0.3 meq/g, and in particular above 0.4 meq/g, are preferred. The amine number denotes, in this context, the milliequivalent amine per gram of the aminofunctional silicone. It can be ascertained by titration, and also indicated using the unit of “mg KOH/g”.

Agents preferred according to the present invention are characterized in that they include, based on their weight, 0.01 to 10 wt %, preferably 0.1 to 8 wt %, particularly preferably 0.25 to 7.5 wt %, and in particular 0.5 to 5 wt % aminofunctional silicone(s).

The cyclic dimethicones referred to according to INCI as Cyclomethicones are also usable with preference according to the present invention. Agents according to the present invention that include at least one silicone of formula Si-III

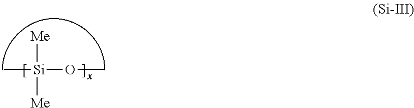

in which x denotes a number from 3 to 200, preferably from 3 to 10, more preferably from 3 to 7, and in particular 3, 4, 5, or 6, are preferred here.

The silicones described above comprise a backbone that is constructed from —Si—O—Si— units. These —Si—O—Si— units can of course also be interrupted by carbon chains. Corresponding molecules are accessible via chain lengthening reactions, and are employed preferably in the form of silicone-in-water emulsions.

Agents likewise preferred according to the present invention are characterized in that they include at least one silicone of formula (Si-IV)

R₃Si—[O—SiR₂]ₓ—(CH₂)ₙ—[O—SiR₂]_{y}—O—SiR₃ (Si-IV),

in which R denotes identical or different residues from the group —H, phenyl, benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y respectively denote a number from 0 to 200, preferably from 0 to 10, more preferably from 0 to 7, and in particular 0, 1, 2, 3, 4, 5, or 6, and n denotes a number from 0 to 10, preferably from 1 to 8, and in particular 2, 3, 4, 5, 6.

The silicones are preferably water-soluble. Agents preferred according to the present invention are characterized in that they include at least one water-soluble silicone.

For aesthetic reasons, “clear” products are often preferred by consumers. Hair treatment agents preferred according to the present invention are therefore characterized in that they are transparent or translucent.

“Transparent or translucent” is understood in the context of the present invention as a composition that has an NTU value below 100. The NTU value (nephelometric turbidity unit) is a unit used in water treatment for turbidity measurements in liquids. It is the unit of the turbidity of a liquid measured with a calibrated nephelometer.

In a preferred embodiment of the invention, an agent according to the present invention can furthermore also include UV filters (I). The UV filters to be used according to the present invention are not subject to any general restrictions in terms of their structure and their physical properties. Instead, all UV filters usable in the cosmetics sector, whose absorption maximum lies in the UVA (315 to 400 nm), UVB (280 to 315 nm), or UVC (<280 nm) regions, are suitable. UV filters having an absorption maximum in the UVB region, in particular in the region from approximately 280 to approximately 300 nm, are particularly preferred.

The UV filters used according to the present invention can be selected, for example, from substituted benzophenones, p-aminobenzoic acid esters, diphenylacrylic acid esters, cinnamic acid esters, salicylic acid esters, benzimidazoles, and o-aminobenzoic acid esters.

Examples of UV filters usable according to the present invention are 4-aminobenzoic acid, N,N,N-trimethyl-4-(2-oxoborn-3-ylidenemethyl)aniline methyl sulfate, 3,3,5-trimethylcyclohexyl salicylate (Homosalate), 2-hydroxy-4-methoxybenzophenone (Benzophenone-3; Uvinul® M 40, Uvasorb® MET, Neo Heliopan®BB, Eusolex® 4360), 2-phenylbenzimidazole-5-sulfonic acid and potassium, sodium, and triethanolamine salts thereof (phenylbenzimidazolesulfonic acid; Parsol® HS; Neo Heliopan® Hydro), 3,3′-(1,4-phenylenedimethylene)-bis(7,7-dimethyl-2-oxo-bicyclo-[2.2.1]hept-1-yl-methanesulfonic acid) and salts thereof, 1-(4-tert-butylphenyl)-3-(4-methoxyphenyl)propane-1,3-dione (butylmethoxydibenzoylmethane; Parsol® 1789, Eusolex® 9020), α-(2-oxoborn-3-ylidene)toluene-4-sulfonic acid and salts thereof, ethoxylated 4-aminobenzoic acid ethyl ester (PEG-25 PABA; Uvinul® P 25), 4-dimethylaminobenzoic acid 2-ethylhexyl ester (Octyl Dimethyl PABA; Uvasorb® DMO, Escalol® 507, Eusolex® 6007), salicylic acid 2-ethylhexyl ester (Octyl Salicylate; Escalol® 587, Neo Heliopan® OS, Uvinul® 018), 4-methoxycinnamic acid isopentyl ester (Isoamyl p-Methoxycinnamate; Neo Heliopan® E 1000), 4-methoxycinnamic acid 2-ethylhexyl ester (Octyl Methoxycinnamate; Parsol® MCX, Escalol® 557, Neo Heliopan® AV), 2-hydroxy-4-methoxybenzophenone-5-sulfonic acid and the sodium salt thereof (Benzophenone-4; Uvinul® MS 40; Uvasorb® S 5), 3-(4′-methylbenzylidene) D,L-camphor (4-Methylbenzylidene Camphor; Parsol® 5000, Eusolex® 6300), 3-benzylidene camphor (3-Benzylidene Camphor), 4-isopropylbenzyl salicylate, 2,4,6-trianilino-(p-carbo-2′-ethylhexyl-1′-oxi)-1,3,5-triazine, 3-imidazol-4-ylacrylic acid and ethyl esters thereof, polymers of N-{(2 and 4)-[2-oxoborn-3-ylidenemethyl]benzyl}acrylamide, 2,4-dihydroxybenzophenone (Benzophenone-1; Uvasorb® 20 H, Uvinul® 400), 1,1′-diphenylacrylonitrilic acid 2-ethylhexyl ester (Octocrylene; Eusolex® OCR, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodiumsulfonate, and 2-cyano-3,3-diphenylacrylic acid 2′-ethylhexyl ester. 4-Aminobenzoic acid, N,N,N-trimethyl-4-(2-oxoborn-3-ylidenemethyl)aniline methyl sulfate, 3,3,5-trimethylcyclohexyl salicylate, 2-hydroxy-4-methoxybenzophenone, 2-phenylbenzimidazole-5-sulfonic acid and potassium, sodium, and triethanolamine salts thereof, 3,3′-(1,4-phenylenedimethylene)-bis(7,7-dimethyl-2-oxobicyclo-[2.2.1]hept-1-ylmethanesulfonic acid) and salts thereof, 1-(4-tert-butylphenyl)-3-(4-methoxyphenyl)propane-1,3-dione, α-(2-oxoborn-3-ylidene)toluene-4-sulfonic acid and salts thereof, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and the sodium salt thereof, 3-(4′-methylbenzylidene) D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-ylacrylic acid and ethyl esters thereof, and polymers of N-{(2 and 4)-[2-oxoborn-3-ylidenemethyl]benzyl}acrylamide are preferred. Very particularly preferred according to the present invention are 2-hydroxy-4-methoxybenzophenone, 2-phenylbenzimidazole-5-sulfonic acid and potassium, sodium, and triethanolamine salts thereof, 1-(4-tert-butylphenyl)-3-(4-methoxyphenyl)propane-1,3-dione, 4-methoxycinnamic acid 2-ethylhexyl ester, and 3-(4′-methylbenzylidene) D,L-camphor.

Those UV filters whose molar extinction coefficient at the absorption maximum is above 15,000, in particular above 20,000, are preferred.

It has furthermore been found that with structurally similar UV filters, in the context of the teaching of the present invention the water-insoluble compound in many cases exhibits the greater effectiveness as compared with those water-soluble compounds which differ from it by having one or more additionally ionic groups. In the context of the invention, those UV filters of which no more than 1 wt %, in particular no more than 0.1 wt %, dissolves in water at 20° C., are understood to be “water-insoluble.” These compounds should furthermore be soluble at a proportion of at least 0.1, in particular at least 1 wt %, in common cosmetic oil components at room temperature. The use of water-insoluble UV filters can therefore be preferred according to the present invention.

According to a further embodiment of the present invention, those UV filters which comprise a cationic group, in particular a quaternary ammonium group, are preferred.

These UV filters exhibit the general structure U-Q.

The structural part U denotes a group that absorbs UV radiation. This group can be derived in principle from the aforementioned known UV filters usable in the cosmetics sector, in which one group, generally a hydrogen atom, of the UV filter is replaced by a cationic group Q, in particular by a quaternary amino function.

Compounds from which the structural part U can be derived are, for example,
substituted benzophenones,
p-aminobenzoic acid esters,
diphenylacrylic acid esters,
cinnamic acid esters,
salicylic acid esters,
benzimidazoles, and
o-aminobenzoic acid esters.

Structural parts U that are derived from cinnamic acid amide or from N,N-dimethylaminobenzoic acid amide are preferred according to the present invention.

The structural parts U can in principle be selected so that the absorption maximum of the UV filters can lie both in the UVA (315 to 400 nm) region and in the UVB (280 to 315 nm) region, or in the UVC (<280 nm) region. UV filters having an absorption maximum in the UVB region, in particular in the region from approximately 280 to approximately 300 nm, are particularly preferred.

The structural part U is furthermore preferably selected, including as a function of the structural part Q, in such a way that the molar extinction coefficient of the UV filter at the absorption maximum is above 15,000, in particular above 20,000.

The structural part Q preferably includes a quaternary ammonium group as a cationic group. This quaternary ammonium group can in principle be connected directly to the structural part U, so that the structural part U represents one of the four substituents of the positively charged nitrogen atom. Preferably, however, one of the four substituents on the positively charged nitrogen atom is a group, in particular an alkylene group, having 2 to 6 carbon atoms, which functions as a connection between the structural part U and the positively charged nitrogen atom.

Advantageously, the group Q has the general structure —(CH₂)_{X}—N⁺R¹R²R³X⁻, in which x denotes an integer from 1 to 4, R¹ and R² mutually independently denote C₁₋₄ alkyl groups, R³ denotes a C₁₋₂₂ alkyl group or a benzyl group, and X⁻ denotes a physiologically acceptable anion. In the context of this general structure, x preferably denotes the number 3, R¹ and R² each denote a methyl group, and R³ denotes either a methyl group or a saturated or unsaturated, linear or branched hydrocarbon chain having 8 to 22, in particular 10 to 18, carbon atoms.

Physiologically acceptable anions are, for example, inorganic anions such as halides, in particular chloride, bromide and fluoride, sulfate ions, and phosphate ions, as well as organic anions such as lactate, citrate, acetate, tartrate, methosulfate, and tosylate.

Two preferred UV filters having cationic groups are the compounds cinnamic acid amidopropyltrimethylammonium chloride (Incroquat® UV-283) and dodecyldimethylaminobenzamidopropyldimethylammonium tosylate (Escalol® HP 610), available as commercial products.

The teaching of the present invention of course also encompasses the use of a combination of several UV filters. In the context of this embodiment, the combination of at least one water-insoluble UV filter with at least one UV filter having a cationic group is preferred.

The UV filters (I) are included in the agents used according to the present invention usually in quantities from 0.1 to 5 wt % based on the entire agent. Quantities from 0.4 to 2.5 wt % are preferred.

The agents according to the present invention can furthermore include a 2-pyrrolidinone-5-carboxylic acid and derivatives thereof (J). The sodium, potassium, calcium, magnesium, or ammonium salts, in which the ammonium ion carries, beside hydrogen, one to three C₁ to C₄ alkyl groups, are preferred. The sodium salt is very particularly preferred. The quantities used in the agents according to the present invention are preferably 0.05 to 10 wt % based on the total agent, particularly preferably 0.1 to 5, and in particular 0.1 to 3 wt %.

It can additionally prove to be advantageous if penetration adjuvants and/or swelling agents (M) are included in the agents according to the present invention. To be included thereamong are, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as 1,2-propanediol, 1,2-pentanediol, 1,2-hexanediol, 1,2-dodecanediol, 1,3-propanediol, 1,6-hexanediol, 1,5-pentanediol, 1,4-butanediol.

In addition, short-chain carboxylic acids (N) can advantageously additionally assist the active agent complex (A). “Short-chain carboxylic acids” and derivatives thereof are understood for purposes of the invention as carboxylic acids that can be saturated or unsaturated and/or straight-chain or branched or cyclic and/or aromatic and/or heterocylic, and have a molecular weight of less than 750. Saturated or unsaturated straight-chain or branched carboxylic acids having a chain length from 1 to 16 carbon atoms in the chain can be preferred for purposes of the invention; those having a chain length from 1 to 12 carbon atoms in the chain are very particularly preferred.

The short-chain carboxylic acids for purposes of the invention can comprise one, two, three, or more carboxy groups. Carboxylic acids having multiple carboxy groups, in particular di- and tricarboxylic acids, are preferred for purposes of the invention. The carboxy groups can be present entirely or partly as an ester, acid anhydride, lactone, amide, imidic acid, lactam, lactim, dicarboximide, carbohydrazide, hydrazone, hydroxam, hydroxime, amidine, amidoxime, nitrile, phosphonic ester, or phosphate ester. The carboxylic acids used according to the present invention can of course be substituted along the carbon chain or along the ring structure. To be included among the substituents of the carboxylic acids used according to the present invention are, for example, C1 to C8 alkyl, C2 to C8 alkenyl, aryl, aralkyl, and aralkenyl, hydroxymethyl, C2 to C8 hydroxyalkyl, C2 to C8 hydroxyalkenyl, aminomethyl, C2 to C8 aminoalkyl, cyano, formyl, oxo, thioxo, hydroxy, mercapto, amino, carboxy, or imino groups. Preferred substituents are C1 to C8 alkyl, hydroxymethyl, hydroxy, amino, and carboxy groups. Substituents in the o-position are particularly preferred. Very particularly preferred substituents are hydroxy, alkoxy, and amino groups, wherein the amino function can optionally be further substituted with alkyl, aryl, aralkyl, and/or alkenyl residues. In addition, the phosphonic esters and phosphate esters are likewise preferred carboxylic acid derivatives.

In a further preferred embodiment the agents according to the present invention can include emulsifier agents (F). Emulsifier agents cause the formation, at the phase interface, of water- or oil-stable adsorption layers that prevent the dispersed droplets from coalescing, and thereby stabilize the emulsion. Emulsifier agents are therefore, like surfactants, constructed from a hydrophobic and a hydrophilic molecule part. Hydrophilic emulsifier agents preferentially form o/w emulsions, and hydrophobic emulsifier agents preferentially form w/o emulsions. An “emulsion” is to be understood as a droplet-like distribution (dispersion) of one liquid in another liquid, with the expenditure of energy to create stabilizing phase interfaces by means of surfactants. Selection of these emulsifying surfactants or emulsifier agents is based on the substances to be dispersed and the respective external phase, and on the fineness of the emulsion. Emulsifier agents usable according to the present invention are, for example:
addition products of 4 to 30 mol ethylene oxide and/or 0 to 5 mol propylene oxide with linear fatty alcohols having 8 to 22 carbon atoms, with fatty acids having 12 to 22 carbon atoms, and with alkylphenols having 8 to 15 carbon atoms in the alkyl group;
C₁₂ to C₂₂ fatty acid mono- and diesters of addition products of 1 to 30 mol ethylene oxide with polyols having 3 to 6 carbon atoms, in particular with glycerol;
addition products of ethylene oxide and polyglycerol with methyl glucoside fatty acid esters, fatty acid alkanolamides, and fatty acid glucamides;
C₈ to C₂₂ alkyl mono- and oligoglycosides and ethoxylated analogs thereof, where degrees of oligomerization from 1.1 to 5, in particular 1.2 to 2.0, and glucose as the sugar component, are preferred;
mixtures of alkyl(oligo)glucosides and fatty alcohols, for example the commercially available product Montanov® 68;
addition products of 5 to 60 mol ethylene oxide with castor oil and hardened castor oil;
partial esters of polyols having 3 to 6 carbon atoms with saturated fatty acids having 8 to 22 carbon atoms;
Sterols. “Sterols” are understood as a group of steroids that carry a hydroxyl group on the third carbon atom of the steroid structure and are isolated both from animal tissue (zoosterols) and from vegetable fats (phytosterols). Examples of zoosterols are cholesterol and lanosterol. Examples of suitable phytosterols are ergosterol, stigmasterol, and sitosterol. Sterols called “mycosterols” are also isolated from fungi and yeasts.
Phospholipids. These are understood principally as the glucose phospholipids, which are obtained e.g. as lecithins or phosphatidylcholines from, for example, egg yolk or plant seeds (e.g. soybeans).
fatty acid esters of sugars and sugar alcohols, such as sorbitol,
polyglycerols and polyglycerol derivatives such as, for example, polyglycerol poly-12-hydroxystearate (commercial product Dehymuls® PGPH),
linear and branched fatty acids having 8 to 30 carbon atoms, and the sodium, potassium, ammonium, calcium, magnesium, and zinc salts thereof.

The agents according to the present invention include the emulsifier agents preferably in quantities from 0.1 to 25 wt %, in particular 0.5 to 15 wt %, based on the entire agent.

The compositions according to the present invention can preferably include at least one nonionogenic emulsifier agent having an HLB value from 8 to 18. Nonionogenic emulsifier agents having an HLB value from 10 to 15 can be particularly preferred according to the present invention.

It has proven to be additionally advantageous if the agents according to the present invention include further polymers, preferably anionic and/or nonionic polymers.

The anionic polymers (G2) are anionic polymers that comprise carboxylate groups and/or sulfonate groups. Examples of anionic monomers of which such polymers can be made up are acrylic acid, methacrylic acid, crotonic acid, maleic acid anhydride, and 2-acrylamido-2-methylpropanesulfonic acid. The acid groups can be present in this context entirely or partially as a sodium, potassium, ammonium, mono- or triethanolammonium salt.

Preferred monomers are 2-acrylamido-2-methylpropanesulfonic acid and acrylic acid.

Anionic polymers that include 2-acrylamido-2-methylpropanesulfonic acid as the only monomer or co-monomer have proven to be very particularly effective, in which context the sulfonic acid group can be present entirely or partly as a sodium, potassium, ammonium, mono- or triethanolammonium salt.

The homopolymer of 2-acrylamido-2-methylpropanesulfonic acid that is obtainable commercially, for example under the designation Rheothik® 11-80, is particularly preferred.

Within this embodiment it can be preferred to employ copolymers of at least one anionic monomer and at least one nonionogenic monomer. With regard to the anionic monomers, reference is made to the substances discussed above. Preferred nonionogenic monomers are acrylamide, methacrylamide, acrylic acid esters, methacrylic acid esters, itaconic acid mono- and diesters, vinylpyrrolidinone, vinyl ethers, and vinyl esters.

Preferred anionic copolymers are acrylic acid/acrylamide copolymers and, in particular, polyacrylamide copolymers having sulfonic acid group-including monomers. A particularly preferred anionic copolymer is made up of 70 to 55 mol % acrylamide and 30 to 45 mol % 2-acrylamide-2-methylpropanesulfonic acid, the sulfonic acid group being present entirely or in part as a sodium, potassium, ammonium, or mono- or triethanolammonium salt. This copolymer can also be present in crosslinked fashion, wherein polyolefinically unsaturated compounds such as tetraallyloxyethane, allylsucrose, allylpentaerythritol, and methylene bisacrylamide are preferably employed as crosslinking agents. One such polymer is included in the commercial product Sepigel® 305 of the SEPPIC company. The use of this compound, which besides the polymer component includes a hydrocarbon mixture (C₁₃ to C₁₄ isoparaffin) and a nonionic emulsifier agent (Laureth-7), has proven particularly advantageous in the context of the teaching of the present invention.

The sodium acryloyl dimethyl taurate copolymers marketed, under the designation Simulgel® 600, as a compound with isohexadecane and polysorbate-80 have also proven particularly effective according to the present invention.

Similarly preferred anionic homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

Polymers of maleic acid anhydride and methyl vinyl ether, in particular those having crosslinks, are also preferred color-retaining polymers. A maleic acid/methyl vinyl ether copolymer crosslinked with 1,9-decadiene is obtainable commercially under the name Stabileze® QM.

In a further embodiment the agents according to the present invention can include nonionogenic polymers (G4).

Suitable nonionogenic polymers are, for example:
Vinylpyrrolidone/vinyl ester copolymers such as those marketed, for example, under the trademark Luviskol® (BASF). Luviskol® VA 64 and Luviskol® VA 73, which are each vinylpyrrolidone/vinyl acetate copolymers, are likewise preferred nonionic polymers.
Cellulose ethers such as hydroxypropyl cellulose, hydroxyethyl cellulose, and methylhydroxypropyl cellulose, such as those marketed, for example, under the trademarks Culminal® and Benecel® (AQUALON) and Natrosol® grades (Hercules).
Starch and derivatives thereof, in particular starch ethers, for example Structure® XL (National Starch), a multifunctional, salt-tolerant starch,
shellac,
polyvinylpyrrolidones such as those marketed, for example, under the designation Luviskol® (BASF),
Siloxanes. These siloxanes can be both water-soluble and water-insoluble. Both volatile and nonvolatile siloxanes are suitable, “nonvolatile siloxanes” being understood as those compounds whose boiling point is above 200° C. at standard pressure. Preferred siloxanes are polydialkylsiloxanes, for example polydimethylsiloxane, polyalklyarylsiloxanes such as polyphenylmethylsiloxane, ethoxylated polydialkylsiloxanes, and polydialkylsiloxanes that include amine and/or hydroxy groups.
Glycosidically substituted silicones.

It is also possible according to the present invention for the preparations to include multiple, in particular two, different polymers of identical charge, and/or respectively one ionic and one amphoteric and/or nonionic polymer.

The further polymers (G) are included in the agents according to the present invention preferably in quantities from 0.05 to 10 wt % based on the total agent. Quantities from 0.1 to 5, in particular from 0.1 to 3 wt %, are particularly preferred.

With regard to further optional components, as well as the quantities of those components used, reference is made expressly to the relevant manuals known to one skilled in the art.

As already mentioned, the excellent care-providing effect of the agents according to the present invention is significant in particular because it provides outstanding results even in the presence of oxidizing agents, for example in the context of oxidative hair coloring.

A further subject of the present invention is a method for treating keratinic fibers, in particular human hair, in which method a hair treatment agent according to the present invention is applied onto keratinic fibers and either left there until the hair is next washed (so-called “leave-on” product) or rinsed out after a contact time from 30 to 300 seconds (so-called “rinse-off” product).

The following examples are intended to explain the subject matter of the present invention without, however, limiting it.

### EXAMPLES

### Hair Rinses

### All Indications in Wt. %

| | Sp-1 | Sp-2 | Sp-3 | Sp-4 | Sp-5 |
|---|---|---|---|---|---|
| Quaternium-91 | 1.3 | 3.5 | 4.0 | 2.5 | 3.0 |
| Tego Amid S 18 | 1.3 | 1.5 | 2.0 | 0.3 | 0.3 |
| Dehyquart F 75 | 1.0 | 3.8 | 0.8 | 1.0 | 1.0 |
| Polyquaternium-37 | 0.5 | 0.4 | 0.75 | 0.2 | 0.1 |
| Octanoic acid cocalkyl | 0.5 | 0.5 | 0.5 | 0.5 | 0.5 |
| ester | | | | | |
| Stenol 1618 | 3.0 | — | — | — | — |
| Cutina GMS-V | — | 0.3 | 0.3 | 0.2 | 0.2 |
| Lanette O | — | 4.0 | 4.0 | 3.0 | 3.0 |
| Eumulgin B2 | — | 0.2 | 0.2 | 0.3 | 0.3 |
| Paraffinum Liquidum | — | 0.8 | 0.8 | | |
| Varisoft W 575 PG | — | | | 4.0 | 4.0 |
| Dow Corning 200, | 1.0 | 1.0 | 0.5 | 1.5 | 0.3 |
| 60000 cSt | | | | | |
| Dow Corning 200 Fluid, | — | 0.5 | 0.5 | 0.3 | 0.1 |
| 0.65 cSt | | | | | |
| Isopropyl myristate | — | — | — | 1.0 | 1.0 |
| Citric acid | — | — | — | 0.4 | 0.4 |
| D-panthenol | 0.2 | — | — | 0.2 | 0.2 |
| Lactic acid | | 0.5 | 0.5 | — | — |
| Methylparaben | 0.2 | — | — | — | — |
| Phenoxyethanol | 0.4 | 0.4 | 0.4 | 0.3 | 0.3 |
| Ectoin | — | 0.1 | 0.3 | 0.3 | 0.3 |
| Taurine | — | 0.1 | 0.3 | 0.5 | 0.3 |
| Perfume | 0.3 | 0.2 | 0.2 | 0.1 | 0.2 |
| Water | to 100 | to 100 | to 100 | to 100 | to 100 |

Hair Therapies

| | K-1 | K-2 | K-3 | K-4 | K-5 | K-6 |
|---|---|---|---|---|---|---|
| Quaternium-91 | 3.0 | 2.5 | 1.0 | 1.5 | 2.0 | 4.0 |
| Tegoamid S 18 | 1.75 | 0.75 | 0.5 | 1.0 | 1.0 | 1.5 |
| Dehyquart F 75 | 2.0 | 1.0 | 0.6 | 0.6 | 0.6 | 0.6 |
| Polyquaternium-37 | 0.75 | 0.6 | 0.5 | 0.4 | 0.3 | 0.75 |
| Octanoic acid cocalkyl ester | 1.0 | — | 0.5 | 0.4 | 0.2 | 0.1 |
| Stenol 1618 | 6.0 | 5.0 | — | — | — | — |
| Gafquat 755 N | — | — | 0.5 | 0.5 | 0.5 | 0.5 |
| Quaternium-80 | — | — | 0.5 | | 0.5 | 1.0 |
| Dow Corning 200, 60000 cSt | — | — | 1.0 | 0.5 | 1.5 | 0.3 |
| Dow Corning 200 Fluid, 0.65 cSt | — | — | 0.5 | 0.5 | 0.3 | 0.1 |
| Ethanol 96% | — | — | 17.0 | 17.0 | 17.0 | 17.0 |
| D-panthenol 75% | 0.5 | — | 0.2 | 0.2 | 0.2 | 0.2 |
| Nicotinic acid amide | — | — | 0.1 | 0.1 | 0.1 | 0.1 |
| Ectoin | — | — | 0.1 | 0.2 | 0.3 | 0.5 |
| Taurine | — | — | 0.1 | 0.2 | 0.3 | 0.5 |
| Methylparaben | 0.2 | 0.1 | — | — | — | — |
| Phenoxyethanol | 0.4 | 0.4 | — | — | — | — |
| Perfume | 0.3 | 0.2 | — | — | — | — |
| Water | to 100 | to 100 | to 100 | to 100 | to 100 | to 100 |

The commercial products employed in the context of the examples are defined as follows:

| | |
|---|---|
| Tego Amid ® S 18 | Dimethyl-N′-stearoyl-1,3-diaminopropane-N,N-stearic |
| | acid-N-(3-dimethylamino)propylamide, fatty acid |
| | amidopropyl betaine (active substance approx. 90 wt %; |
| | INCI name: Stearamidopropyl Dimethylamine) |
| | (Goldschmidt) |
| Dehyquart ® F75 | Fatty alcohol-methyltriethanolammonium methyl sulfate- |
| | dialkyl ester mixture (INCI name: Distearoylethyl |
| | Hydroxyethylmonium Methosulfate, Cetearyl Alcohol) |
| | (BASF) |
| Stenol ® 1618 | C₁₆₋₁₈ fatty alcohol (INCI name: Cetearyl Alcohol) (Cognis) |
| Cutina ® GMS-V | Glycerol mono-/dipalmitate/stearate (INCI name: Glyceryl |
| | Stearate) (Cognis) |
| Lanette ® O | C₁₆₋₁₈ fatty alcohol (INCI name: Cetearyl Alcohol) (Cognis) |
| Eumulgin ® B2 | Cetylstearyl alcohol with approx. 20 EO units (INCI name: |
| | Ceteareth-20) (Cognis) |
| Varisoft ® W 575 PG | INCI name: Quaternium-87 (Goldschmidt) |
| Dow Corning ® 200 fluid | Polydimethylsiloxane (INCI name: Dimethicone) (Dow |
| | Corning) |
| Gafquat ® 755 | Dimethylaminoethyl methacrylate/vinylpyrrolidone |
| | copolymer, quaternized with diethyl sulfate (approx. 19% |
| | solids in water; INCI name: Polyquaternium-11) (ISP). |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent, comprising:
a) at least one quaternary imidazoline derivative having at least two long fat residues in accordance with formula I 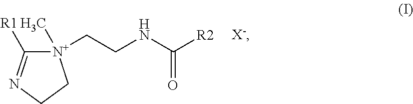
wherein the residues R¹ and R² mutually independently each denote a saturated or unsaturated, linear or branched hydrocarbon residue having a chain length from 18 to 30 carbon atoms, and X⁻ denotes an anion,
b) at least one amine and/or cationized amine,
c) at least one esterquat, and
d) poly(2-methacryloxyethyltrimethylammonium chloride) (INCI name: Polyquaternium-37).

2. The hair treatment agent according to claim 1, characterized in that it additionally includes esters of coconut fatty alcohols with octanoic acid.

3. The hair treatment agent according to claim 1, characterized in that it includes, based on its weight, 0.1 to 15 wt % of at least one quaternary imidazoline derivative of formula I in which each of R¹ and R² denotes —(CH₂)₂₀—CH₃ and X⁻ denotes CH₃—OSO₃⁻.

4. The hair treatment according to claim 1, characterized in that it includes, based on its weight, 0.1 to 15 wt % dimethyl-N′-stearoyl-1,3-diaminopropane (INCI name: Stearamidopropyl Dimethylamine).

5. The hair treatment agent according to claim 1, characterized in that it includes, based on its weight, 0.1 to 15 wt % distearoylethylhydroxyethylmethylammonium methosulfate (INCI name: Distearoylethyl Hydroxyethylmonium Methosulfate).

6. The hair treatment agent according to claim 1, characterized in that it further includes, based on its weight, 0.1 to 15 wt % poly(2-methacryloxyethyltrimethylammonium chloride) (INCI name: Polyquaternium-37).

7. The hair treatment agent according to claim 1, characterized in that it further includes, based on its weight, 0.01 to 5 wt % cetyltrimethylammonium chloride.

8. The hair treatment agent according to claim 1, characterized in that it further includes, based on its weight, 0.01 to 5 wt % cetearyl alcohol.

9. The hair treatment agent according to claim 1 including, based on its weight,
a) 0.5 to 2.5 wt % of at least one quaternary imidazoline derivative of formula I in which R¹ and R² each denote —(CH₂)₂₀—CH₃ and X⁻ denotes CH₃—OSO₃⁻,
b) 0.5 to 2.5 wt % dimethyl-N′-stearoyl-1,3-diaminopropane (INCI name: Stearamidopropyl Dimethylamine),
c) 0.5 to 2.5 wt % distearoylethylhydroxyethylmethylammonium methosulfate (INCI name: Distearoylethyl Hydroxyethylmonium Methosulfate),
d) 0.5 to 2.5 wt % poly(2-methacryloxyethyltrimethylammonium chloride) (INCI name: Polyquaternium-37).

10. The hair treatment agent according to claim 1 including, based on its weight,
a) 0.5 to 2.5 wt % of at least one quaternary imidazoline derivative of formula I in which R¹ and R² each denote —(CH₂)₂₀—CH₃ and X⁻ denotes CH₃—OSO₃⁻,
b) 0.5 to 2.5 wt % dimethyl-N′-stearoyl-1,3-diaminopropane (INCI name: Stearamidopropyl Dimethylamine),
c) 0.5 to 2.5 wt % distearoylethylhydroxyethylmethylammonium methosulfate (INCI name: Distearoylethyl Hydroxyethylmonium Methosulfate),
d) 0.5 to 2.5 wt % poly(2-methacryloxyethyltrimethylammonium chloride) (INCI name: Polyquaternium-37),
e) 0.1 to 10 wt % esters of coconut fatty alcohols with octanoic acid.
